(19) United States
(12) Patent Application Publication
(10) Pub. No.: US 2024/0165384 A1
(43) Pub. Date: May 23, 2024

(54) Tattoo Machine With Lighting

(71) Applicant: FK Irons Inc., Doral, FL (US)

(72) Inventors: Gaston Alfonso Siciliano, Bal Harbour, FL (US); Fernando Diaz, Miami, FL (US); Alex Moribe, Pembroke Pines, FL (US)

(73) Assignee: FK Irons Inc., Doral, FL (US)

(21) Appl. No.: 18/389,510

(22) Filed: Nov. 14, 2023

Related U.S. Application Data
(60) Provisional application No. 63/384,355, filed on Nov. 18, 2022.

(51) Int. Cl. A61M 37/00

(52) U.S. Cl. CPC A61M 37/0076; A61M 2205/12; A61M 2205/3306; A61M 2205/3375; A61M 2205/3592; A61M 2205/52; A61M 2205/587; A61M 2205/8262

(57) ABSTRACT

A tattoo device comprises a machine section, a power pack, and a light emitter. The machine section comprises a working end and a power-receiving end on a side other than the working end. The working end is configured to receive a needle or needle cartridge that facilitates tattooing a working surface. The power pack is configured to couple to the power receiving end of the machine section. The power pack comprises a first section comprising a power interface configured to couple to the power receiving end of the machine section, and a second section having a longitudinal axis that is parallel with and offset from a longitudinal axis of the first section and that extends along at least a portion of the machine section in a direction towards the working end. The light emitter is coupled to the second section of the power pack.

### BACKGROUND

### Field

This application generally relates to tattoo machines. In particular, this application describes a tattoo machine with integrated and/or detachable lighting and/or cameras.

### Description of Related Art

Tattoo machines are hand-held devices used to mark the skin with ink. Tattoo machines typically comprise an electromechanical device configured to cause a needle to move in a reciprocating manner against the skin to push ink into the skin. The reciprocating action of the needle and relative ease with which the tattoo machine can be maneuvered allows a tattoo artist to creatively apply ink to the skin.

### SUMMARY

In a first aspect, a tattoo device comprises a machine section, a power pack, and a light emitter. The machine section comprises a working end and a power-receiving end. The working end is configured to receive a needle or needle cartridge that facilitates tattooing a working surface. The power pack is configured to couple to the power receiving end of the machine section. The power pack comprises a first section comprising a power interface configured to couple to the power receiving end of the machine section, and a second section having a longitudinal axis that is parallel with and offset from a longitudinal axis of the first section and that extends along at least a portion of the machine section in a direction towards the working end. The light emitter is coupled to the second section of the power pack.

In a second aspect, a tattoo device comprises a machine section, a power adapter, and an arm member. The machine section comprises a working end and a power-receiving end. The working end is configured to receive a needle or needle cartridge that facilitates tattooing a working surface. The power adapter is configured to couple to the power receiving end of the machine section. The power adapter comprises a connector configured to couple to a complementary connector of a power cable or power pack. The arm member comprises a light emitter positioned at a distal end. The arm member comprises a light emitter, a first connector, and a second connector. The light emitter is positioned at a distal end of the arm member. The first connector configured to be removably coupled to the connector of the power adapter. The second connector is configured to be removably coupled to the complementary connector of the power cable.

In a third aspect, a power pack for a tattoo device comprises a first section, a second section, and a light emitter that is coupled to the second section. The first section comprises a power interface configured to couple to a power receiving end of a tattoo device. The second section has a longitudinal axis that is parallel with and offset from a longitudinal axis of the first section, and that is configured to extend along at least a portion of a machine section of the tattoo device in a direction towards a working end of the tattoo device when the power pack is coupled to the tattoo device. The light emitter is configured to illuminate the working surface that is in front of the working end of the tattoo device.

In a fourth aspect, an arm member comprises a light emitter, a first connector, and a second connector. The light emitter is positioned at a distal end of the arm member. The first connector is configured to be removably coupled to a connector of a power adapter of a tattoo device, and the second connector is configured to be removably coupled to a connector of a power cable that is configured to be removably coupled to the connector of the power adapter of the tattoo device.

### BRIEF DESCRIPTION OF THE DRAWINGS

The accompanying drawings are included to provide a further understanding of the claims, are incorporated in, and constitute a part of this specification. The detailed description and illustrated examples described serve to explain the principles defined by the claims.

FIG. 1A illustrates a perspective view of a tattoo device that includes a light emitter at the distal end of an arm member, in accordance with example embodiments.

FIG. 1B illustrates a side view of the tattoo device, in accordance with example embodiments.

FIG. 1C illustrates rotation of the arm member, in accordance with example embodiments.

FIG. 2A illustrates a first side view of a tattoo device that includes an arm member that is configured to slide longitudinally along the tattoo device, in accordance with example embodiments.

FIG. 2B illustrates a second side view of the tattoo device, in accordance with example embodiments.

FIG. 3A illustrates a side view of a tattoo device that includes a rotatable arm member that is configured to slide longitudinally along the tattoo device, in accordance with example embodiments.

FIG. 3B illustrates a top view of the tattoo device, in accordance with example embodiments.

FIG. 4 illustrates a side view of a tattoo device that includes an articulating arm, in accordance with example embodiments.

FIG. 5 illustrates a side view of a tattoo device that includes a telescoping arm, in accordance with example embodiments.

FIG. 6 illustrates a side view of a tattoo device that includes a pair of light emitters at respective distal ends of a pair of arm members, in accordance with example embodiments.

FIG. 7 illustrates a side view of a tattoo device that includes a light emitter and an imager at the distal end of an arm member, in accordance with example embodiments.

FIG. 8 illustrates various devices that can be removably coupled to a power pack of a tattoo device to facilitate illuminating a working surface, in accordance with example embodiments.

FIG. 9 illustrates a side view of a tattoo device that includes a power adapter and an illuminating device configured to be coupled to the power adapter, in accordance with example embodiments.

FIG. 10 illustrates a side view of a tattoo device that includes a power adapter and an illuminating device configured to be coupled to the power adapter, and that includes an articulating arm member, in accordance with example embodiments.

### DETAILED DESCRIPTION

Various examples of devices are described herein. Words such as “example” that may be used herein are understood to mean “serving as an example, instance, or illustration.” Any embodiment, implementation, and/or feature described herein as being an “example” is not necessarily to be construed as preferred or advantageous over any other embodiment, implementation, and/or feature unless stated as such. Thus, other embodiments, implementations, and/or features may be utilized, and other changes may be made without departing from the scope of the subject matter presented herein.

Accordingly, the examples described herein are not meant to be limiting. It will be readily understood that the aspects of the present disclosure, as generally described herein, and illustrated in the figures, can be arranged, substituted, combined, separated, and designed in a wide variety of different configurations.

Further, unless the context suggests otherwise, the features illustrated in each of the figures may be used in combination with one another. Thus, the figures should be generally viewed as component aspects of one or more overall embodiments, with the understanding that not all illustrated features are necessary for each embodiment.

Moreover, terms such as “substantially” or “about” that may be used herein, are meant that the recited characteristic, parameter, or value need not be achieved exactly, but that deviations or variations, including, for example, tolerances, measurement error, measurement accuracy limitations and other factors known to skill in the art, may occur in amounts that do not preclude the effect the characteristic was intended to provide.

### I. Introduction

As noted above, tattoo machines typically include an electromechanical device configured to cause a needle to move in a reciprocating manner against the skin to push ink into the skin. The reciprocation action of the needle and relative ease with which the tattoo machine can be maneuvered allows a tattoo artist to creatively apply ink to the skin. The process of applying the artwork can be time-consuming. For example, it may take a tattoo artist several hours to apply even relatively simple tattoos. During this time, the tattoo artist is working up close to the customer (e.g., within a few inches) and maneuvering around the customer.

Lighting is an important consideration. For example, the tattoo artist may use a powerful light at the end of a light stand to illuminate the working surface. However, the light may have to be repositioned from time to time to avoid casting shadows. This may frustrate the process of applying the ink.

These and other issues are ameliorated by various examples of tattoo devices disclosed herein. For instance, an example of a tattoo device includes a machine section and a power pack 110. The machine section includes a working end and a power receiving end on a side other than the working end. In some examples, the power receiving end is on a side that is opposite the working end. The working end is configured to receive a needle or needle cartridge that facilitates tattooing a working surface (e.g., skin). The power pack is configured to couple to the power receiving end of the machine section 105. An example of the power pack includes a first section and a second section. The first section includes a power interface that is configured to couple to the power receiving end of the machine section 105. The second section has a longitudinal axis that is parallel with and offset from a longitudinal axis of the first section, and that extends along at least a portion of the machine section in a direction towards the working end. A light emitter that is configured to illuminate the working surface is coupled to the second section.

Some examples of the tattoo device include an arm member. In some examples, the arm member is coupled to the second section, and the light emitter is positioned at the distal end of the arm member. The distal end of the arm member is configured to direct light emitted from the light emitter at the working surface. In some examples, the arm member is rotatably coupled to an end of the second section to facilitate rotation of the arm member and, therefore, the light emitter about the longitudinal axis of the second section. In some examples, the arm member is slidably coupled to the second section to facilitate sliding the arm member and, therefore, the light emitter along the longitudinal axis of the second section. In some examples, the arm member is both slidably and rotatably coupled to the second section to facilitate sliding the arm member and, therefore, the light emitter along the longitudinal axis of the second section and rotating the arm member about an axis that passes through a longitudinal axis of the machine section 105.

In some examples, the distal end of the arm member includes an imager that is configured to capture one or more images of the working surface. In this regard, some examples of the power pack include a transceiver configured to communicate captured images to a receiving device, such as a smart device, computer, etc. Some examples of the power pack are configured to receive a removable data storage device and to store captured images on the removable data storage device.

Some examples of the tattoo device include an articulating member, and the articulating member is coupled to the second section. In these examples, the light emitter is positioned at the distal end of the articulating member. In some examples, the articulating member includes an articulating housing, and a light pipe is positioned within the articulating housing. The light pipe is configured to communicate light that is emitted from an illuminating device within the power pack to the light emitter. Some examples of the tattoo device include a telescoping arm. The telescoping arm is coupled to the second section, and the light emitter is positioned at the distal end of the telescoping arm.

In some examples of the tattoo device, the various arm members described above are removable and interchangeable.

### II. Example Tattoo Device

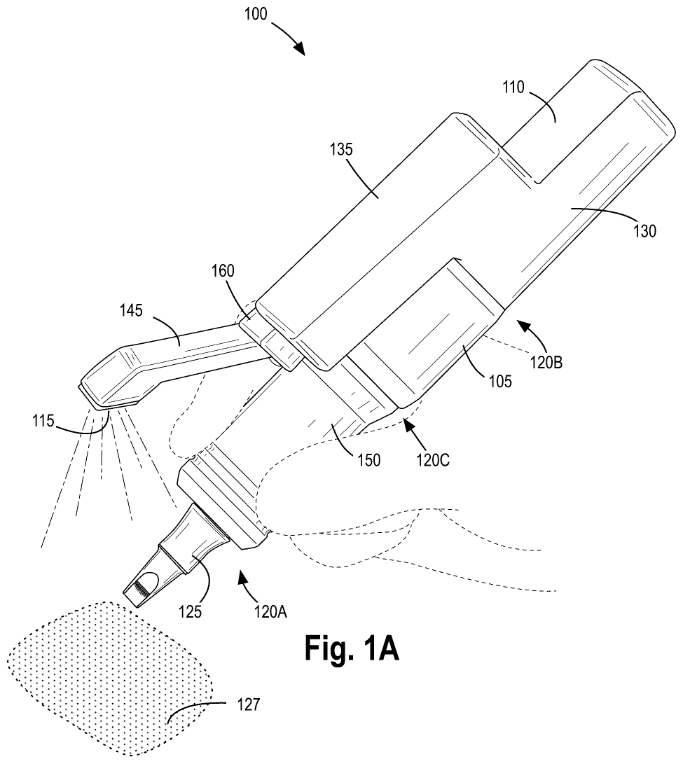

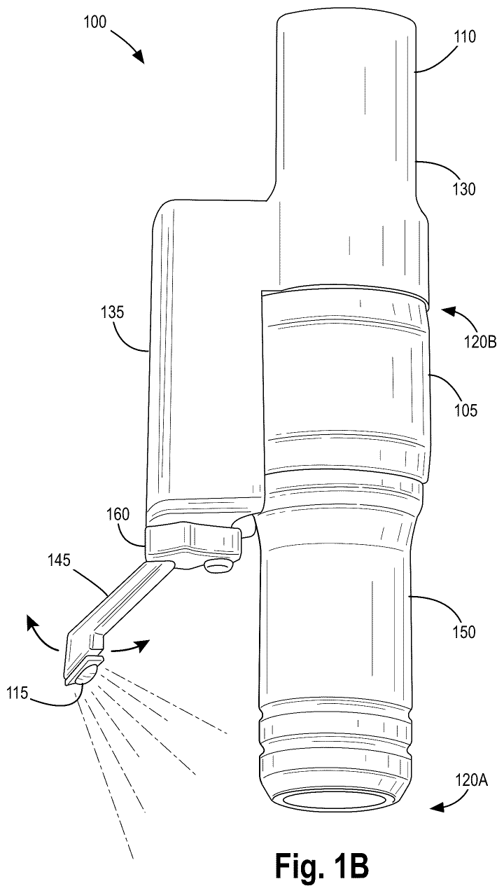

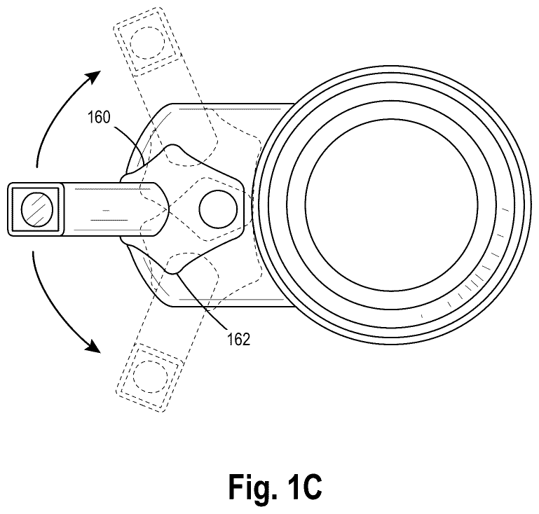

FIGS. 1A-1B illustrate various views of an example tattoo device 100. The tattoo device 100 includes a machine section 105, a power pack 110, and a light emitter 115. An example of the machine section 105 includes a working end 120A and a power receiving end 120B. The working end 120A of the example tattoo device 100 is shown as being on a side that is opposite the working end 120A. In other example tattoo devices, the working end is on a different side of the tattoo device.

The working end 120A is configured to receive a needle cartridge 125 that includes a needle (or in some cases a plurality of needles) and that facilitates tattooing a working surface 127. Some examples of the working end 120A are additionally, or alternatively, configured to receive a needle directly (i.e., without requiring a needle cartridge).

Some examples of the machine section 105 include a motor assembly configured to receive power via the power receiving end 120B and from the power pack 110 to drive the needle of the needle cartridge 125. Some examples of the motor assembly are configured to cause one or more needles of the needle cartridge 125 to move in a reciprocating manner along, for example, the longitudinal axis of the machine section 105. In some examples, the rate at which and/or the amount by which the needle reciprocates is adjustable.

Some examples of the machine section 105 have a somewhat elongated cylindrical shape. In this regard, in some examples, the machine section 105 includes a gripping region 150 that extends from a middle region 120C of the machine section 105 towards the working end 120A of the machine section 105 and a rearward region (e.g., a section between the middle region 120C and the power receiving end 120B). Some examples of the rearward region have a generally cylindrical shape. However, other examples of the rearward region can have a rectangular shape, multifaceted shape, etc.

Some examples of the gripping region 150 have a somewhat tapered shape (e.g., tapering towards the working end 120A) to facilitate comfortably gripping the working end 120A of the machine section 105. In some examples, the outer surface of the gripping region 150 is covered by a rubberized material to enhance the gripping characteristics of the gripping region 150. Some examples of the outer surface may define one or more grooved regions that further enhance the gripping characteristics of the gripping region 150. In some examples, the grooves may be evenly spaced around the gripping region 150.

In some examples, the motor described above is housed within the rearward region of the machine section 105, and one or more gears, shafts, etc., extend through the gripping region 150 and engage the needle. In some examples, circuitry is provided within the machine section 105 to control the operation of the motor (e.g., the speed of the motor). Some examples of the circuitry are configured to ensure the voltage received from the power pack 110 is communicated to the motor in a consistent manner. For instance, an example of the circuitry is configured to rectify the voltage so that the polarity of the voltage is consistent. An example of the circuitry includes voltage regulation circuitry configured to regulate the voltage communicated to the motor.

An example of the power pack 110 is configured to be coupled to the power receiving end 120B of the machine section 105. In this regard, an example of the power pack 110 includes a first section 130 and a second section 135. Some examples of the first section 130 of the power pack 110 and/or the power receiving end 120B of the machine section 105 include one or more features that facilitate removably coupling/securing the power pack 110 to the power receiving end 120B of the machine section 105. For example, the first section 130 of the power pack 110 and/or the power receiving end 120B of the machine section 105 may include or define features that facilitate inserting a portion of the power pack 110 into the power receiving end 120B of the machine section 105 and rotating the power pack 110 with respect to the power receiving end 120B to lock the power pack 110 to the power receiving end 120B of the machine section 105. In another example, the features facilitate inserting a portion of the power receiving end 120B of the machine section 105 into a complementary portion of the first section 130 of the power pack 110 to rotatably lock the power pack 110 to the power receiving end 120B of the machine section 105. In yet another example, one or both of the first section 130 of the power pack 110 and/or the power receiving end 120B of the machine section 105 includes magnets (e.g., rear earth magnets) that facilitate releasable and magnetically coupling the power pack 110 to the power receiving end 120B of the machine section 105.

In some examples, the first section 130 and the second section 135 correspond to first and second housing sections of a housing of the power pack 110. An example of the first section 130 has an elongated shape. In some examples, the longitudinal axis of the first section 130 is aligned with the longitudinal axis of the machine section 105. An example of the first section 130 has a somewhat cuboid shape (e.g., four elongated sidewalls that are perpendicular to one another). Examples of the first section 130 can be cylindrically shaped, multifaceted, etc.

An example of the second section 135 has an elongated shape. In some examples, the longitudinal axis of the second section 135 is offset from the longitudinal axis of the first section 130 of the power pack 110 to allow the second section 135 to overlap a portion of the machine section 105. An example of the second section 135 has a somewhat cuboid shape (e.g., four elongated sidewalls that are perpendicular to one another). Examples of the second section 135 can be cylindrically shaped, multifaceted, etc.

In some examples, a battery (e.g., rechargeable battery) is positioned within the power pack 110. For instance, the battery may be positioned within the first section 130 or the second section 135 of the power pack 110. In some examples, a plurality of batteries (e.g., two batteries) are positioned within the power pack 110. For instance, a first battery may be positioned in the first section 130, and a second battery may be positioned in the second section 135. The batteries may be electrically coupled in series or in parallel with one another. In some examples, the power pack 110 includes two or more terminals that are configured to electrically couple terminals of the battery or batteries to corresponding terminals (or PCB contact pads) of the machine section 105 to provide power to the motor.

As noted above, some examples of the machine section 105 include a gripping region 150 that extends from the middle region 120C of the machine section 105 toward the working end 120A of the machine section 105. In some examples, the second section 135 of the power pack 110 extends up to about the middle region 120C so as not to frustrate the gripping of the machine section 105 by a user.

### III. Tattoo Device Lighting

As noted above, some examples of tattoo devices include a light emitter 115. In this regard, some examples of the light emitter 115 are coupled to the second section 135 of the power pack 110 and are configured to illuminate the working surface 127. Some examples of the light emitter 115 correspond to an illuminating device, such as a light-emitting diode (LED). In some examples, an illuminating device is positioned within the power pack 110 (e.g., within the second section 135 of the power pack 110), and light from the illuminating device light is optically communicated to the light emitter 115 via, for example, a light pipe. In some examples, circuitry configured to drive the illuminating device is provided within the power pack 110. An example of the circuitry is configured to provide a fixed or variable voltage for driving the illuminating device. For example, a variable voltage may be provided to facilitate adjustment of the intensity of light emitted by the illuminating device. In this regard, some examples of the power pack 110 may include a user interface (e.g., up/down buttons, knob, etc.) that facilitates adjusting the intensity of light emitted by the illuminating device.

In some examples, light emitted by the illuminating device corresponds to white light. In some examples, the illuminating device corresponds to a multicolored LED, and the color or color temperature of the light emitted by the illuminating device can be changed (e.g., to red, green, blue, etc.) to facilitate emphasizing portions of a tattoo that are drawn in a particular color. In this regard, some examples of the tattoo device include user controls that facilitate adjusting the lighting characteristics. Some examples of the tattoo device include wireless communication circuitry (e.g., a Bluetooth® transceiver) that facilitates adjusting the lighting characters via, for example, an application operating on a smart device (e.g., smartphone, smart tablet, etc.).

### IV. First Example Arm Member Implementation

As noted above, some examples tattoo device 100 include an arm member 145. Some examples of the arm member 145 extend from the power pack 110, and the light emitter 114 is positioned at the distal end of the arm member 145. For example, an LED may be provided at the distal end of the arm member 145, and conductors may run from the LED and through the arm member 145 to circuitry in the power pack 110 configured to provide power to the LED. In some examples, an illuminating device is positioned within the power pack 110 (e.g., within the second section 135 of the power pack), and a light pipe extends from the illuminating device through the arm member 145 and to the distal end of the arm member 145.

In some examples, the arm member 145 extends from an end of the second section 135 that is proximate to the middle region 120C (e.g., where the gripping region 150 of the machine section 105 begins) to or toward the working end 120A of the machine section 105. In some examples, the arm member 145 extends along the longitudinal axis of the second section 135 so that the arm member 145 extends somewhat parallel to the machine section 105. In some examples, to minimize interference with the user's fingers, the arm member 145 is angled to an extent (e.g., by about 30 degrees) so that the distance between the arm member 145 and the gripping region 150 of the machine section 105 gradually increases when moving towards the distal end of the arm member 145.

As shown in FIG. 1C, in some examples, the arm member 145 is rotatably coupled to the power pack 110 (e.g., the second section 135 of the power pack 110). This configuration facilitates rotation of the light emitter 115 about, for example, the longitudinal axis of the second section 135 of the power pack 110. In this regard, in some examples, the arm member 145 includes a rotatable coupler 160 that, in turn, is coupled to the power pack 110. In some examples, the rotatable coupler 160 is configured to limit the amount of rotation. For example, as shown in FIG. 1C, the rotatable coupler 160 may be configured to limit the rotation of the arm member 145 to about ±45. In this regard, some examples of the rotatable coupler 160 include features 162 configured to act as stops that limit the amount of rotation.

### V. Second Example Arm Member Implementation

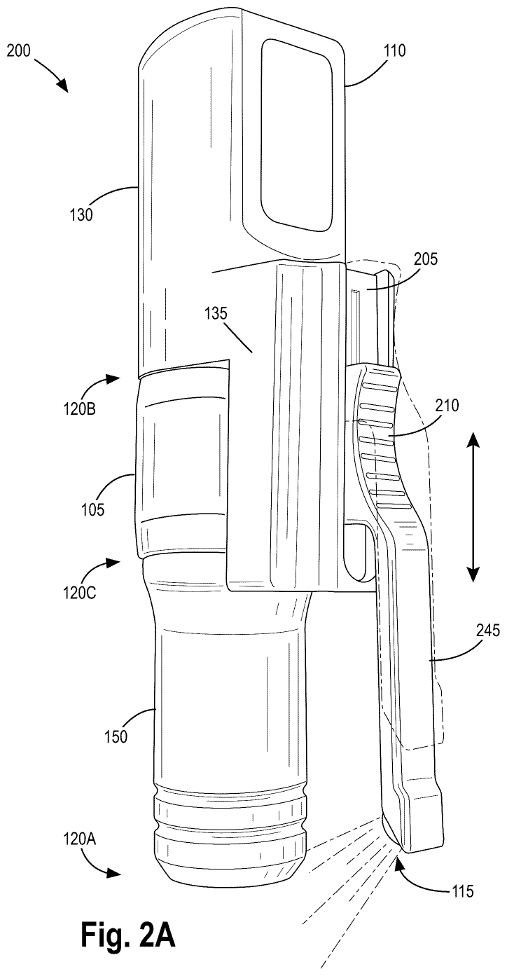

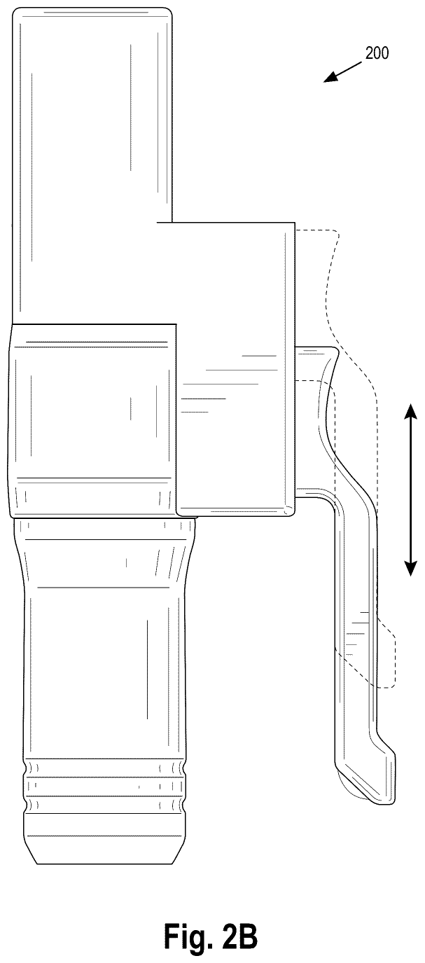

FIGS. 2A and 2B illustrate side views of an example tattoo device 200 that includes a light emitter at the distal end of an arm member 245 that is configured to slide longitudinally along the tattoo device 200. The tattoo device 200 includes a machine section 105, a power pack 110, and a light emitter 115. Examples of the machine section 105, power pack 110, and light emitter 115 include one or more of the features and/or are configured to perform one or more of the operations of the corresponding devices described above.

For example, the power pack 110 of the tattoo device includes a first section 130 and a second section 135. In some examples, the longitudinal axis of the second section 135 is offset from the longitudinal axis of the first section 130 of the power pack 110. In some examples, the light emitter 115 is positioned at the distal end of the arm member 245.

Some examples of the second section 135 include a channel 205 that is configured to receive the proximate end 210 of the arm member 245 and to allow the proximate end 210 of the arm member 245 to slide within the channel 205. Some examples of the channel 205 are configured to allow the proximate end 210 of the arm member 245 to slide along a portion of the machine section 105 that spans between approximately the power receiving end 120B of the machine section 105 and the middle region 120C of the machine section 105.

Some examples of the arm member 245 extend along the longitudinal axis of the second section 135 so that the arm member 145 extends somewhat parallel to the machine section 105. Some examples of the arm member 245 may be angled to an extent (e.g., by about 30 degrees) so that the distance between the arm member 145 and the gripping region 150 of the machine section 105 gradually increases when moving towards the distal end of the arm member 245. This, in turn, may minimize interference between the user's fingers and the arm member 245.

Some examples of the proximate end 210 of the arm member 245 define an inwardly curved feature sized to facilitate pushing the arm member 245 (e.g., using the thumb) along the longitudinal axis of the second section 135. Some examples of the curved feature define grooves or ridges that increase friction between the thumb and the proximate end 210 of the arm member 245 to mitigate slippage of the thumb along that arm member 245 that may otherwise occur when attempting to push the arm member 245 along the longitudinal axis of the second section 135.

In some examples, one or both of the channel 205 of the second section 135 and the proximate end 210 of the arm member 245 define detent features that facilitate locking or maintaining the arm member 245 within one of a plurality of detent positions (e.g., ten detent positions) along the channel 205. Pushing or pulling of the proximate end 210 (e.g., using the thumb) can move the proximate end 210 and, therefore, the arm member 245 between the detent positions.

### VI. Third Example Arm Member Implementation

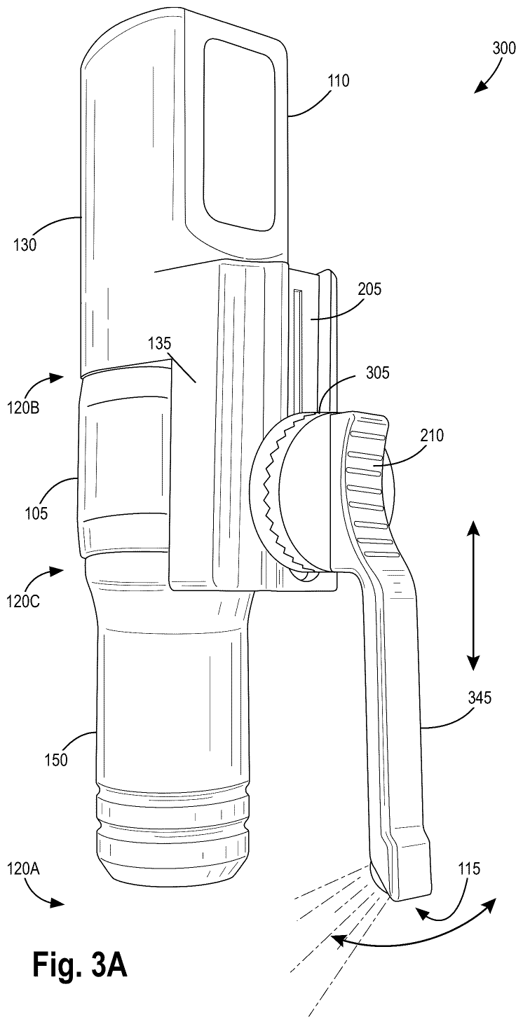

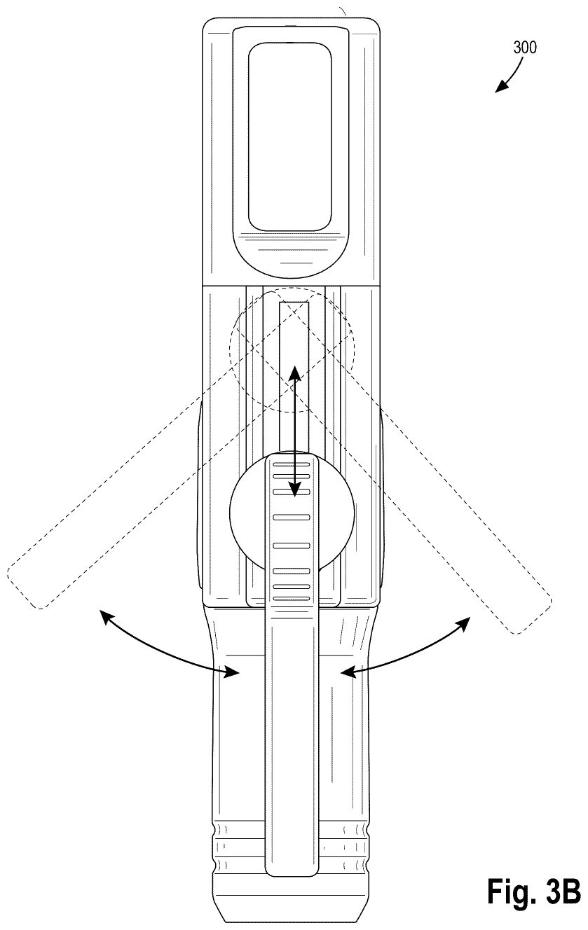

FIGS. 3A and 3B illustrate side views of an example tattoo device 300 that includes a light emitter 115 at the distal end of a rotatable arm member 345 that is configured to slide longitudinally along the tattoo device 300. The tattoo device 300 includes a machine section 105, a power pack 110, and a light emitter 115. Examples of the machine section 105, power pack 110, and light emitter 115 include one or more of the features and/or are configured to perform one or more of the operations of the corresponding devices described above.

For example, some examples of the second section 135 include a channel 205 that is configured to receive the proximate end 210 of the arm member 345 and to allow the proximate end 210 of the arm member 345 to slide within the channel 205. Some examples of the arm member 345 extend along the longitudinal axis of the second section 135 so that the arm member 345 extends somewhat parallel to the machine section 105. Some examples of the proximate end 210 of the arm member 345 define an inwardly curved feature sized to facilitate pushing the arm member 245 (e.g., using the thumb) along the longitudinal axis of the second section 135 of the power pack 110.

Some examples of the arm member 345 include a coupler 305 that provides two degrees of freedom of movement for the arm member 345. For instance, some examples of the coupler 305 facilitate longitudinal movement of the arm member 345 within the channel 205 and rotational movement of the arm member 345 about an axis that passes through a longitudinal axis of the machine section 105. Additionally, some examples of coupler 305 define detent features that facilitate locking or maintaining the arm member 345 within one of a plurality of detent positions along the channel 205 and one of a plurality of rotational positions. The two degrees of freedom gives the user more freedom to reposition the light emitter 115 as needed.

### VII. Fourth Example Arm Member Implementations

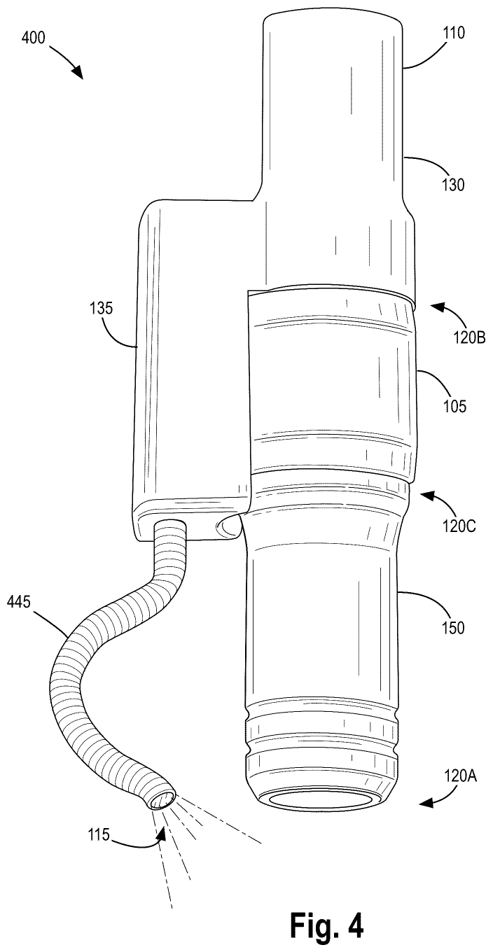

FIG. 4 illustrates a side view of a tattoo device 400 that includes a light emitter 115 at the distal of an articulating arm 445. The tattoo device 300 includes a machine section 105, a power pack 110, and a light emitter 115. Examples of the machine section 105, power pack 110, and light emitter 115 include one or more of the features and/or are configured to perform one or more of the operations of the corresponding devices described above.

For example, some examples of the articulating arm 445 extend from an end of the second section 135 of the power pack 110 that is proximate to the middle region 120C (e.g., where the gripping region 150 of the machine section 105 begins). The articulating arm 445 is configured to be articulated to facilitate directing light that is emitted from the light emitter 115 in a variety of directions. In some examples, the length of the articulating arm 445 is selected to allow the user to bend a region of the articulating arm 445 nearest the gripping region 150 around and away from the gripping region 150 to increase the distance between the gripping region 150 of the machine section 105 and the articulating arm 445, thus minimizing interference between the user's hand and the articulating arm 445.

Some examples of the articulating arm 445 correspond to a hollow articulating tubular material. In some examples, the light emitter 115 corresponds to an LED or similar device and conductors for powering the light emitter 115 extend through hollow articulating tubular material. In some examples, an illuminating device is positioned within the power pack 110 (e.g., within the second section 135 of the power pack 110), and light from the illuminating device light is communicated to the light emitter 115 via, for example, a light pipe that extends through the hollow articulating tubular material.

### VIII. Fifth Example Arm Member Implementation

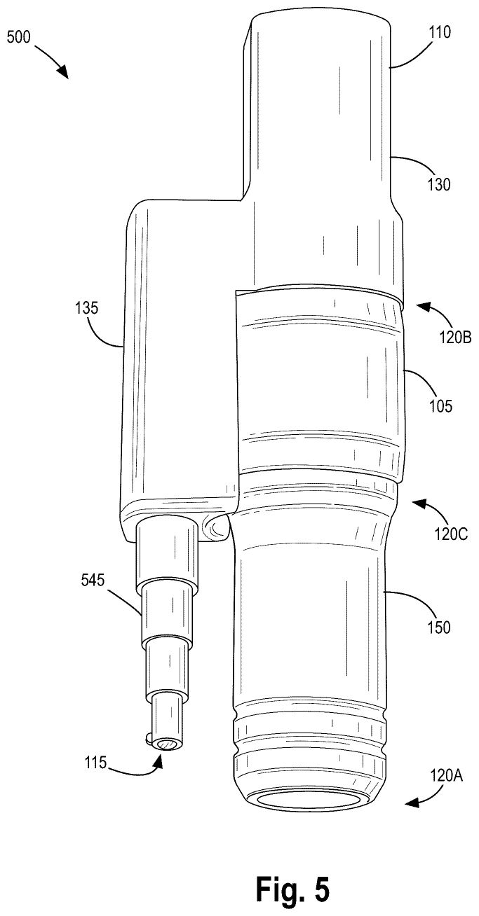

FIG. 5 illustrates a side view of a tattoo device 500 that includes a light emitter 115 at the distal of a telescoping arm 545. The tattoo device 300 includes a machine section 105, a power pack 110, and a light emitter 115. Examples of the machine section 105, power pack 110, and light emitter 115 include one or more of the features and/or are configured to perform one or more of the operations of the corresponding devices described above.

For example, some examples of the telescoping arm 545 extend from an end of the second section 135 of the power pack 110 that is proximate to the middle region 120C (e.g., where the gripping region 150 of the machine section 105 begins). The telescoping arm 545 is configured to be telescopically extended to facilitate moving the light emitter 115 to within a desired distance of the working surface 127 (e.g., to broaden or narrow the light incident on the working surface 127).

Some examples of the telescoping arm 545 include several (e.g., three or more) hollow cylindrical segments. In some examples, the light emitter 115 corresponds to an LED or similar device and conductors for powering the light emitter 115 extend through the segments. In some examples, an illuminating device is positioned within the power pack 110 (e.g., within the second section 135 of the power pack 110), and light from the illuminating device light is communicated to the light emitter 115 via, for example, a light pipe that extends through the segments. Some examples of the light pipe correspond to a flexible optical material (e.g., one or more fiber optic cables) that can be retracted to an extent from the second section 135 of the power pack 110 when the telescoping arm 545 is extended.

### IX. Example Tattoo Device with Multiple Arms

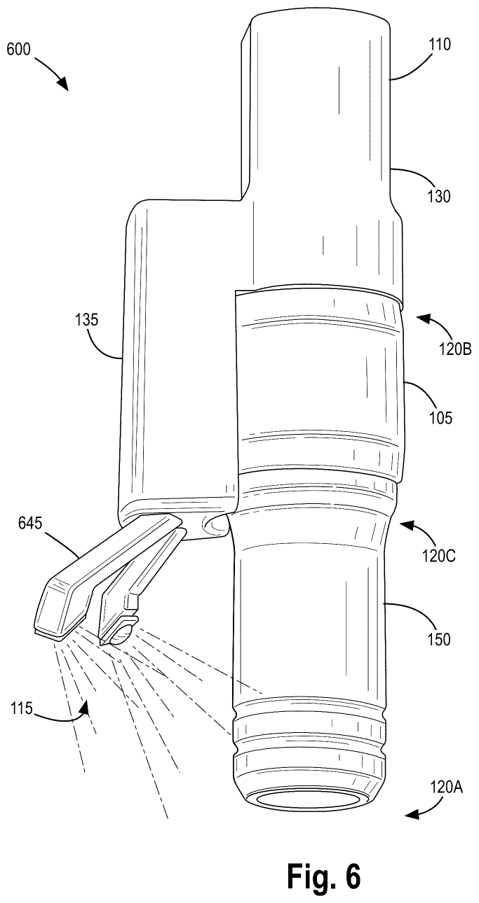

FIG. 6 illustrates a side view of an example of a tattoo device 600 that includes multiple light emitters 115 at respective distal ends of multiple arm members 645. The tattoo device 600 shown in the figure includes a pair arm members 645 and light emitters 115, but other examples of the tattoo device can include additional arm members 645 and light emitters 115. The tattoo device 300 includes a machine section 105, a power pack 110, and a light emitter 115. Examples of the machine section 105, power pack 110, and light emitter 115 include one or more of the features and/or are configured to perform one or more of the operations of the corresponding devices described above.

For example, some examples of the pair of arm members 645 extend from the power pack 110. In some examples, conductors may extend through the arm members 645 to, for example, LEDs positioned at respective distal ends of the arm members 645. In some examples, light pipes may extend through the arm members 645 to communicate light from an illuminating device positioned within the power pack 110 to the respective light emitters 115.

Some examples of the pair of arm members 645 are angled to an extent (e.g., by about 30 degrees) to increase space between the gripping region 150 and the arm member 645. Some examples of the pair of arm members 645 are rotatably coupled to the power pack 110 (e.g., the second section 135 of the power pack 110) to facilitate rotation of the respective light emitter 115 about, for example, the longitudinal axis of the second section 135 of the power pack 110. In this regard, some examples of the pair of arm members 645 include a rotatable coupler 160 that is, in turn, coupled to the power pack 110. In some examples, the rotatable coupler 160 is configured to limit the amount of rotation.

### X. Example Tattoo Device with an Integrated or Detachable Camera

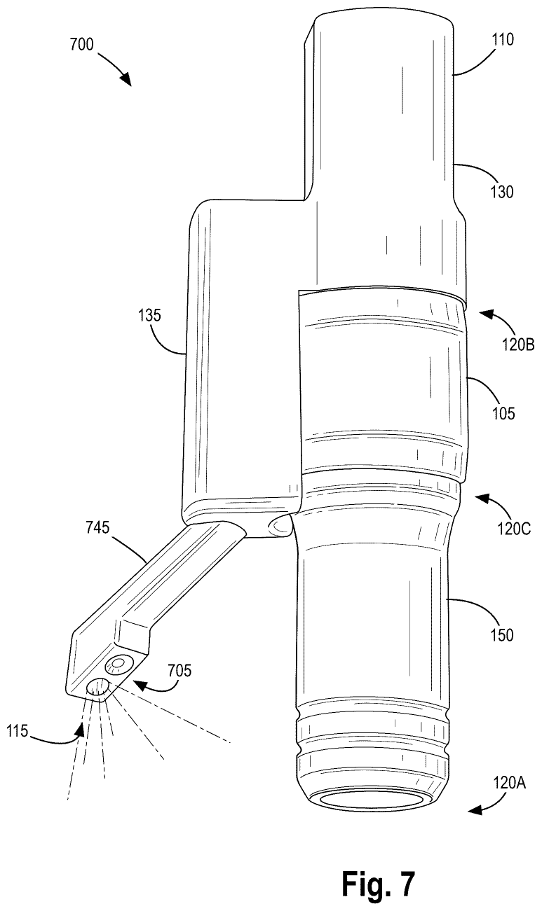

FIG. 7 illustrates a side view of a tattoo device 700 that includes a light emitter 115 and an imager 705 at the distal end of an arm member 745. The tattoo device 300 includes a machine section 105, a power pack 110, and a light emitter 115. Examples of the machine section 105, power pack 110, and light emitter 115 include one or more of the features and/or are configured to perform one or more of the operations of the corresponding devices described above.

For example, some examples of the arm member 745 extend from the power pack 110. In some examples, conductors may extend through the arm member 745 to, for example, LEDs positioned at the distal end of the arm member 745 and the imager 705. In some examples, a light pipe may extend through the arm member 745 to communicate light from an illuminating device positioned within the power pack 110 to the light emitter 115.

Some examples of the arm members 745 are angled to an extent (e.g., by about 30 degrees) to increase space between the gripping region 150 and the arm member 745. Some examples of the arm member 745 are rotatably coupled to the power pack 110 (e.g., the second section 135 of the power pack 110) to facilitate rotation of the respective light emitter 115 about, for example, the longitudinal axis of the second section 135 of the power pack 110. In this regard, some examples, the arm members 745 include a rotatable coupler 160 that is, in turn, coupled to the power pack 110. In some examples, the rotatable coupler 160 is configured to limit the amount of rotation.

Some examples of the imager 705 are configured to capture an image and/or video content that depicts the working surface 127 as the user operates the tattoo device 700. Some examples of the tattoo device include one or more microphones configured to capture audio content. Captured images, video content, and audio content can be communicated to remote devices.

In this regard, some examples of the power pack 110 include a transceiver configured to communicate the images and/or audio/video content to a receiving device. For instance, some examples of the power pack 110 include a Bluetooth®, WiFi®, or other similarly capable transceiver configured to wirelessly communicate the images and/or audio/video content to remote devices that are similarly equipped. Some examples of the transceiver are configured to compress the images and/or audio/video content to reduce the bandwidth required to communicate the images and/or audio/video content. Some examples of the transceiver can be configured to perform other image/video operations. For instance, some examples of the operations facilitate adjusting the exposure and color temperature of the image/video. Some examples of the operations facilitate adjusting the framerate and resolution of the image/video.

Some examples of the power pack 110 are configured to receive a removable data storage device (e.g., a micro secure digital (SD) card) configured to store captured images, video content, and/or audio content. Some examples of the power pack 110 include a wired data interface (e.g., universal serial bus (USB) interface) that facilitates wired communication to a remote device.

### XI. Example Tattoo Device with Removable Illuminating Devices

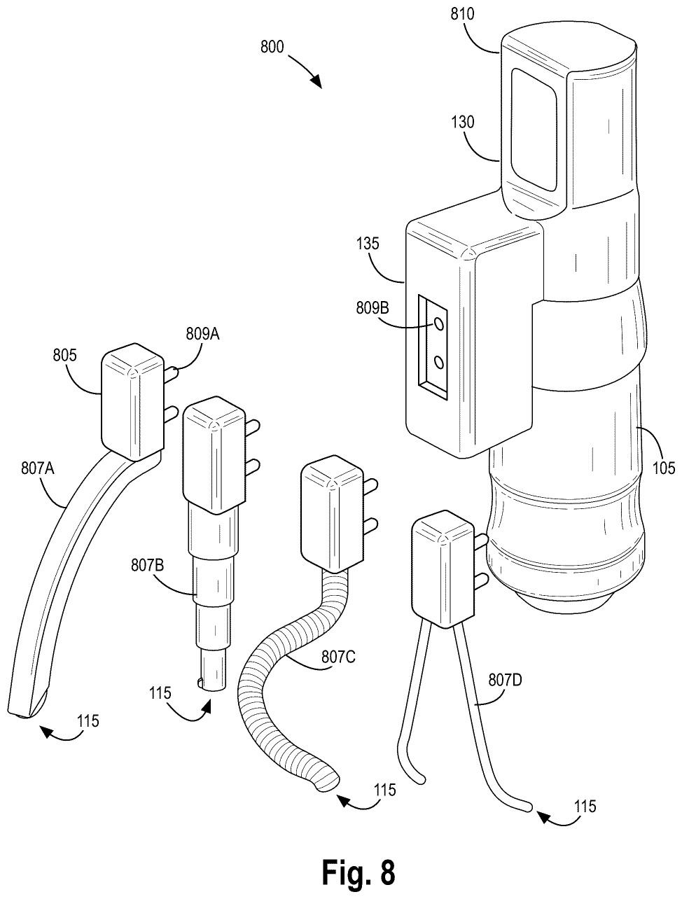

FIG. 8 illustrates examples of various devices 807 that can be removably coupled to the power pack 110 of a tattoo device 800 to facilitate illuminating a working surface 127. The tattoo device 800 includes a machine section 105 and a power pack 810. Examples of the machine section 105 and the power pack 840 include one or more of the features and/or are configured to perform one or more of the operations of the corresponding devices described above.

Some examples of the devices 807 include an arm member and a coupler 805. For instance, a first example device 807A includes an arm member having features and capabilities similar to the arm member 145 described in FIG. 1A. A second example device 809B includes an arm member having features and capabilities similar to the arm member 245 described in FIG. 2A. A third example device 807C includes an arm member having features and capabilities similar to the articulating arm 445 described in FIG. 4. A fourth example device 807D includes a pair of arm members having features and capabilities similar to the arm members 645 described in FIG. 6.

Some examples of the coupler 805 include features that are configured to engage complementary features of the power pack 110 to facilitate removably coupling the devices 807 to the power pack 110. For instance, some examples of the coupler 805 include one or more protrusions 809A configured to enter corresponding openings 809B defined in the second section 135 of the power pack 110. In some examples, a pair of protrusions 809A are used to prevent the rotation of the coupler 805. In some examples, the second section 135 defines a recess into which a portion of the coupler extends. In some examples, the respective shapes of the recess and the coupler 805 are configured to prevent rotation of the coupler 805. In some examples, of the coupler 805 and/or power pack 110 include one or more magnets that facilitate magnetically fastening/securing the coupler 805 to the power pack 110.

In some examples, one or more of the protrusions 809A include one or more conductive sections (e.g., one or more conductively coated regions). When the coupler 805 is coupled to the power pack 810, the conductive sections are configured to electrically contact terminals positioned within the power pack 810 that are proximate to the openings 809B. This, in turn, facilitates communicating power to, for example, an LED positioned at the distal end of the arm member of the device 807.

### XII. Example Illuminating Devices for Tattoo Power Adapter

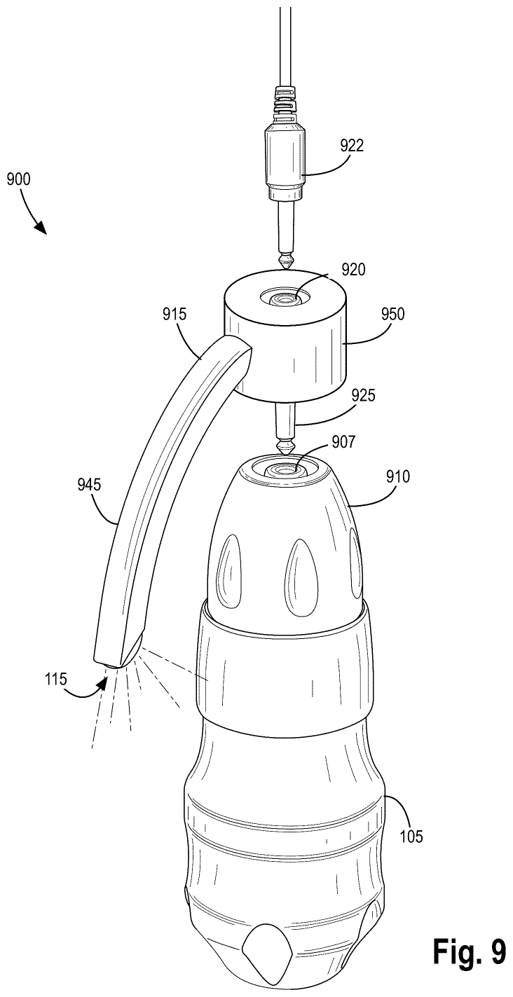

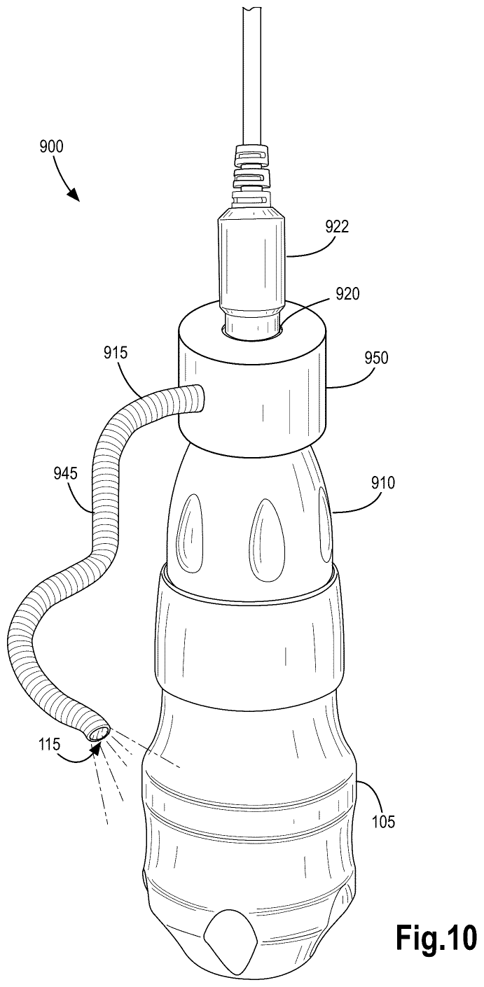

FIGS. 9 and 10 illustrate side views of an example tattoo device 900 that includes a light emitter 115 at the distal and of an arm member 945 that can be removably coupled to a power adapter 910 of the tattoo device 900.

Examples of the machine section 105 include one or more of the features and/or are configured to perform one or more of the operations of machine sections described above. In this regard, some examples of the power receiving end 120B of the machine section 105 are configured to receive power from the power adapter 910 to drive the needle of the needle cartridge 125.

An example of the power adapter 910 is configured to be coupled to the power receiving end 120B of the machine section 105. In this regard, some examples of the power adapter 910 include one or more features that facilitate removably coupling/securing the power adapter 910 to the power receiving end 120B of the machine section 105. For example, the power adapter 910 and/or the power receiving end 120B of the machine section 105 may include or define features that facilitate inserting a portion of the power adapter 910 into the power receiving end 120B of the machine section 105 and rotating the power adapter 910 with respect to the power receiving end 120B to lock the power adapter 910 to the power receiving end 120B of the machine section 105. In another example, the features facilitate inserting a portion of the power receiving end 120B of the machine section 105 into a complementary portion of the power adapter 910 to rotatably lock the power adapter 910 to the power receiving end 120B of the machine section 105. In yet another example, one or both of the power adapter 910 and/or the power receiving end 120B of the machine section 105 include magnets (e.g., rear earth magnets) that facilitate releasable magnetically coupling the power adapter 910 to the power receiving end 120B of the machine section 105.

Some examples of the power adapter 910 include a connector 907 configured to mechanically and electrically couple to a complementary connector of a power cable 922 or a power pack. In this regard, some examples of the connector 907 correspond to a ¼″ female (or male) audio jack connector (e.g., a headphone type of connector). Some examples of the connector 907 correspond to an RCA type of connector. Some examples of the connector 907 correspond to a USB connector. Other connectors capable of carrying sufficient current to the machine section 105 to power the motor of the tattoo device 900 can be used.

Some examples of the arm member 945 include a coupler 950 at the proximate end. Examples of the arm member 945 include one or more features and capabilities similar to the arm member 145, described in FIG. 1A, the arm member 245, described in FIG. 2A, the articulating arm 445, described in FIG. 4, etc.

Some examples of the coupler 950 include features that are configured to engage complementary features of the power adapter 910 to facilitate removably coupling the arm member 945 to the power adapter 910. For instance, some examples of the coupler 805 include a first connector 925 (e.g., ¼″ male audio jack) configured to mechanically and electrically couple to the connector 907 of the power adapter 910. Some examples of the coupler 950 include a second connector 920 configured to couple to a power cable 922 and are configured to route power received via the second connector 920 to the first connector 925. That is, the coupler is configured to act as a passthrough device for passing power to the tattoo device 900. In some examples, the light emitter 115 corresponds to an illuminating device and power received via the second connector is also routed (e.g., via conductors) through the arm member 945 to the illuminating device. In some examples, the illuminating device is positioned within the coupler 950, and a light pipe extends from the illuminating device through the arm member 145 and to the light emitter 115 at the distal end of the arm member 945 to illuminate the working surface 127.

While various devices have been described with reference to certain examples, it will be understood by those skilled in the art that various changes can be made and equivalents can be substituted without departing from the scope of the claims. Therefore, it is intended that the claims be limited to the particular examples disclosed.

1. A tattoo device comprising:
a machine section comprising a working end and a power receiving end on a side other than the working end, wherein the working end is configured to receive a needle or needle cartridge that facilitates tattooing a working surface;
a power pack configured to couple to the power receiving end of the machine section, wherein the power pack comprises:
a first section comprising a power interface configured to couple to the power receiving end of the machine section; and
a second section having a longitudinal axis that is parallel with and offset from a longitudinal axis of the first section and that extends along at least a portion of the machine section in a direction towards the working end; and
a light emitter coupled to the second section.

2. The tattoo device according to claim 1, further comprising an arm member coupled to the second section, wherein the light emitter is positioned at a distal end of the arm member, and wherein the distal end of the arm member is configured to direct light emanating from the light emitter at a working surface.

3. The tattoo device according to claim 2, wherein the arm member is rotatably coupled to an end of the second section to facilitate rotation of the light emitter about the longitudinal axis of the second section.

4. The tattoo device according to claim 2, wherein the arm member is slidably coupled to the second section to facilitate sliding the light emitter along the longitudinal axis of the second section.

5. The tattoo device according to claim 2, wherein the arm member is slidably and rotatably coupled to the second section to facilitate sliding the light emitter along the longitudinal axis of the second section and rotating the arm member about an axis that passes through a longitudinal axis of the machine section.

6. The tattoo device according to claim 1, further comprising an articulating member coupled to the second section, wherein the light emitter is positioned at a distal end of the articulating member.

7. The tattoo device according to claim 6, wherein the articulating member comprises an articulating housing and a light pipe within the articulating housing, wherein the light pipe is configured to communicate light that is emitted from a light source within the power pack to the light emitter.

8. The tattoo device according to claim 1, further comprising a telescoping arm coupled to the second section, wherein the light emitter is positioned at a distal end of the telescoping arm.

9. The tattoo device according to claim 1, wherein the machine section comprises a gripping region that extends from a middle region of the machine section towards the working end of the machine section, wherein the second section of the power pack extends up to about the middle region so as not to frustrate gripping of the machine section by a user.

10. The tattoo device according to claim 2, wherein the distal end of the arm member comprises an imager that facilitates capture of one or more images or video of the working surface.

11. The tattoo device according to claim 10, wherein the power pack comprises a transceiver configured to communicate the one or more images to a receiving device.

12. The tattoo device according to claim 10, wherein the power pack is configured to receive a removable data storage device configured to store the one or more images.

13. The tattoo device according to claim 1, further comprising a microphone configured to capture audio content.

14. The tattoo device according to claim 1, wherein the power pack is configured to be releasably coupled to the power receiving end of the machine section.

15. The tattoo device according to claim 2, wherein the arm member is removably coupled to the power pack.

16. A tattoo device comprising:
a machine section comprising a working end and a power receiving end on a side other than the working end, wherein the working end is configured to receive a needle cartridge that facilitates tattooing a working surface;
a power adapter configured to couple to the power receiving end of the machine section, wherein the power adapter comprises a connector configured to couple to a complementary connector of a power cable or of a power pack; and
an arm member that comprises:
a light emitter positioned at a distal end of the arm member;
a first connector configured to be removably coupled to the connector of the power adapter; and
a second connector that is configured to be removably coupled to the complementary connector of the power cable.

17. The tattoo device according to claim 16, wherein the arm member is configured to communicate power received via the complementary connector of the power cable or of the power pack to the connector of the power adapter.

18. The tattoo device according to claim 16, wherein the arm member is configured so that light emitted from the light emitter is directed to the working surface.

19. The tattoo device according to claim 18, wherein the light emitter corresponds to an illuminating device that is configured to receive power via the power cable or the power pack.

20. The tattoo device according to claim 18, wherein the arm member comprises a coupler at a proximate end, wherein the coupler comprises an illuminating device, and wherein a light pipe positioned within the arm member is configured to communicate light emitted by the illuminating device to the light emitter.
